(19) United States
(12) Patent Application Publication
(10) Pub. No.: US 2025/0360260 A1
(43) Pub. Date: Nov. 27, 2025

(54) UNIVERSAL NEGATIVE PRESSURE DEVICE, MOUNT, AND SYSTEM

(71) Applicant: Solventum Intellectual Properties Company, Maplewood, MN (US)

(72) Inventors: Larry T. RANDOLPH, San Antonio, TX (US); Michael R. OLIVA, Saint Paul, MN (US); Richard M. KAZALA, JR., San Antonio, TX (US)

(21) Appl. No.: 18/872,439

(22) PCT Filed: May 31, 2023
(86) PCT No.: PCT/IB2023/055603

§ 371 Date: Dec. 6, 2024

Related U.S. Application Data
(60) Provisional application No. 63/351,053, filed on Jun. 10, 2022.

(51) Int. Cl. A61M 1/00

(52) U.S. Cl. CPC A61M 1/962; A61M 1/92; A61M 2205/3337; A61M 2205/583; A61M 2205/7536; A61M 2209/088

(57) ABSTRACT

A negative pressure therapy assembly for treating a tissue site includes a negative pressure therapy device. The negative pressure therapy device includes a housing defining an enclosure that is hermetically sealed. Additionally, the housing includes a base portion and a cover portion. The negative pressure therapy device also includes at least one housing aperture disposed through the base portion in fluid communication with the enclosure and a negative pressure pump. The negative pressure pump is configured to expose the enclosure to a negative pressure.

### CROSS-REFERENCE TO RELATED APPLICATIONS

This application is a U.S. National Stage Entry of PCT International Application No. PCT/IB2023/055603, filed on May 31, 2023, which claims the benefit of priority to U.S. Provisional Application No. 63/351,053, filed on Jun. 10, 2022, each of which are incorporated herein by reference in their entirety.

### TECHNICAL FIELD

The invention set forth in the appended claims relates generally to tissue treatment systems and more particularly, but without limitation, to a reduced-pressure device with a universal mounting system.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

There is also widespread acceptance that cleansing a tissue site can be highly beneficial for new tissue growth. For example, a wound or a cavity can be washed out with a liquid solution for therapeutic purposes. These practices are commonly referred to as “irrigation” and “lavage” respectively. “Instillation” is another practice that generally refers to a process of slowly introducing fluid to a tissue site and leaving the fluid for a prescribed period of time before removing the fluid. For example, instillation of topical treatment solutions over a wound bed can be combined with negative-pressure therapy to further promote wound healing by loosening soluble contaminants in a wound bed and removing infectious material. As a result, soluble bacterial burden can be decreased, contaminants removed, and the wound cleansed.

While the clinical benefits of negative-pressure therapy and/or instillation therapy are widely known, improvements to therapy systems, components, and processes may benefit healthcare providers and patients.

### BRIEF SUMMARY

New and useful systems, apparatuses, and methods for providing negative-pressure to a tissue site are set forth in the appended claims. Illustrative embodiments are also provided to enable a person skilled in the art to make and use the claimed subject matter.

For example, in some embodiments, a negative pressure therapy assembly for treating a tissue site includes a negative pressure therapy device. The negative pressure therapy device includes a housing defining an enclosure that is hermetically sealed. Additionally, the housing includes a base portion and a cover portion. The negative pressure therapy device also includes at least one housing aperture disposed through the base portion in fluid communication with the enclosure and a negative pressure pump. The negative pressure pump is configured to expose the enclosure to a negative pressure.

In another illustrative example embodiment, a negative pressure therapy assembly for treating a tissue site includes a negative pressure therapy device. The negative pressure therapy assembly includes a housing defining an enclosure that is hermetically sealed. The housing also includes a base portion and a cover portion. Additionally, the negative pressure therapy assembly includes at least one vacuum port, at least one sensing port, and a negative pressure pump. The at least one vacuum port is disposed through the base portion in fluid communication with the enclosure. The at least one sensing port is also disposed in the base portion. The negative pressure pump is disposed in the enclosure and configured to expose the enclosure to a negative pressure.

In yet another example embodiment, a system for treating a tissue site with negative pressure includes a negative pressure therapy device, a mount configured to receive the negative pressure therapy device, and a dressing. The negative pressure therapy device includes a housing defining an enclosure that is hermetically sealed. Additionally, the housing includes a base portion and a cover portion. The negative pressure therapy device also includes at least one vacuum port disposed through the base portion in fluid communication with the enclosure, at least one sensing port disposed in the base portion, and a negative pressure pump disposed in the enclosure. The negative pressure pump is configured to expose the enclosure to the negative pressure. The mount includes a receptacle, a mating surface coupled to the receptacle, and at least one mount aperture disposed in the mating surface. The receptacle is configured to receive at least the base portion of the housing. The mating surface is configured to create a hermetic seal relative to at least a portion of the base portion. Each of the at least one mount apertures is configured to be positioned in fluid communication with one of the at least one vacuum port or one of the at least one sensing port. The dressing is configured to be positioned at the tissue site and in fluid communication with the at least one mount aperture.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a block diagram of an example embodiment of a therapy system that can provide negative-pressure treatment in accordance with this specification;

FIG. 2 is an exploded view of an example embodiment of a negative pressure therapy assembly that may be associated with some embodiments of the therapy system of FIG. 1;

FIG. 3 is an exploded view of a negative pressure therapy device of the negative pressure therapy assembly of FIG. 2, illustrating additional details that may be associated with some embodiments;

FIG. 4 is an exploded view of another example embodiment of a negative pressure therapy assembly that may be associated with some embodiments of the therapy system of FIG. 1;

FIG. 5A is an exploded view of another example embodiment of a negative pressure therapy assembly that may be associated with some embodiments of the therapy system of FIG. 1;

FIG. 5B is a bottom, exploded view of the negative pressure therapy assembly of FIG. 5A, illustrating additional details that may be associated with some embodiments;

FIG. 6 is an exploded view of another example embodiment of a negative pressure therapy assembly that may be associated with some embodiments of the therapy system of FIG. 1;

FIG. 7 is an exploded view of another example embodiment of a negative pressure therapy assembly that may be associated with some embodiments of the therapy system of FIG. 1;

FIG. 8 is an exploded view of a negative pressure therapy device of the negative pressure therapy assembly of FIG. 7, illustrating additional details that may be associated with some embodiments;

FIG. 9A is a top view of a printed circuit board of the negative pressure therapy device of FIG. 8, illustrating additional details that may be associated with some embodiments;

FIG. 9B is a bottom view of the printed circuit board of FIG. 9A, illustrating additional details that may be associated with some embodiments;

FIG. 10 is an exploded view of a mount of the negative pressure therapy assembly of FIG. 7, illustrating additional details that may be associated with some embodiments;

FIG. 11A is an exploded view of another example embodiment of a negative pressure therapy assembly that may be associated with some embodiments of the therapy system of FIG. 1;

FIG. 11B is an exploded view of a canister of the negative pressure therapy assembly of FIG. 11A, illustrating additional details that may be associated with some embodiments;

FIG. 11C is an exploded view of a filter assembly of the canister of FIG. 11A, illustrating additional details that may be associated with some embodiments;

FIG. 11D is a perspective view of a tubing assembly of the canister of FIG. 11A, illustrating additional details that may be associated with some embodiments;

FIG. 12A is an exploded view of another example embodiment of a negative pressure therapy assembly that may be associated with some embodiments of the therapy system of FIG. 1;

FIG. 12B is an exploded view of a dressing transition pad of the negative pressure therapy assembly of FIG. 12A, illustrating additional details that may be associated with some embodiments;

FIG. 13A is an exploded view of another example embodiment of a negative pressure therapy assembly that may be associated with some embodiments of the therapy system of FIG. 1;

FIG. 13B is an exploded view of an absorbent transition pad of the negative pressure therapy assembly of FIG. 13A, illustrating additional details that may be associated with some embodiments;

FIG. 14A is an exploded view of another example embodiment of a negative pressure therapy assembly that may be associated with some embodiments of the therapy system of FIG. 1; and

FIG. 14B is an exploded view of a trauma cup of the negative pressure therapy assembly of FIG. 14A, illustrating additional details that may be associated with some embodiments.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

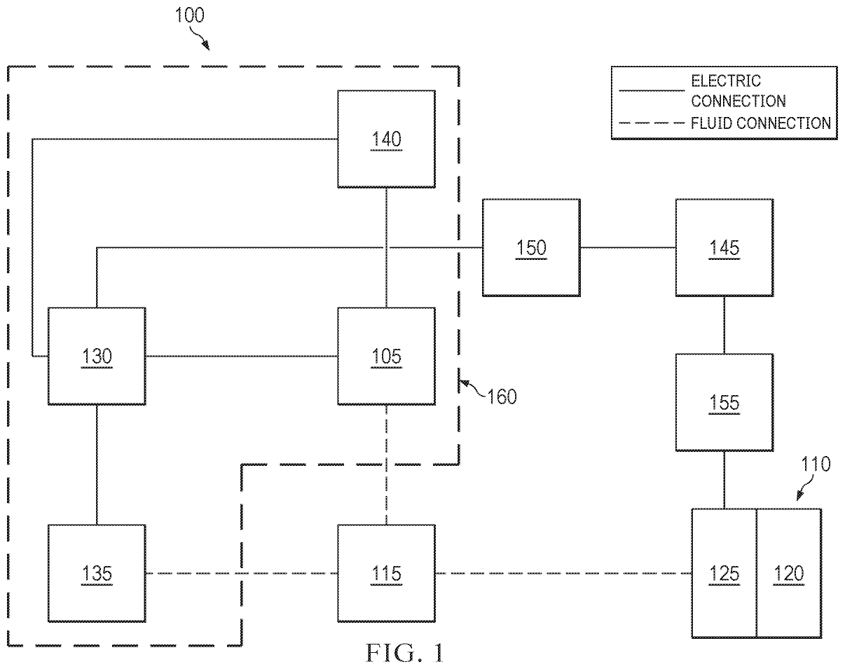

FIG. 1 is a block diagram of an example embodiment of a therapy system 100 that can provide negative-pressure therapy with instillation of topical treatment solutions to a tissue site in accordance with this specification.

The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

The therapy system 100 may include a source or supply of negative pressure, such as a negative-pressure source 105, and one or more distribution components. A distribution component is preferably detachable and may be disposable, reusable, or recyclable. A dressing, such as a dressing 110, and a fluid container or canister, such as a canister 115, are examples of distribution components that may be associated with some examples of the therapy system 100. As illustrated in the example of FIG. 1, the dressing 110 may comprise or consist essentially of a tissue interface 120, a cover 125, or both in some embodiments.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 110. For example, such a dressing interface may be a SENSAT.R.A.C.™ Pad available from Kinetic Concepts, Inc. of San Antonio, Texas.

The therapy system 100 may also include a regulator or controller, such as a controller 130. Additionally, the therapy system 100 may include sensors to measure operating parameters and provide feedback signals to the controller 130 indicative of the operating parameters. As illustrated in FIG. 1, for example, the therapy system 100 may include a first sensor 135 and a second sensor 140 coupled to the controller 130.

The therapy system 100 may also include a source of instillation solution. For example, a solution source 145 may be fluidly coupled to the dressing 110, as illustrated in the example embodiment of FIG. 1. The solution source 145 may be fluidly coupled to a positive-pressure source such as a positive-pressure source 150, a negative-pressure source such as the negative-pressure source 105, or both in some embodiments. A regulator, such as an instillation regulator 155, may also be fluidly coupled to the solution source 145 and the dressing 110 to ensure proper dosage of instillation solution (e.g. saline) to a tissue site. For example, the instillation regulator 155 may comprise a piston that can be pneumatically actuated by the negative-pressure source 105 to draw instillation solution from the solution source during a negative-pressure interval and to instill the solution to a dressing during a venting interval. Additionally or alternatively, the controller 130 may be coupled to the negative-pressure source 105, the positive-pressure source 150, or both, to control dosage of instillation solution to a tissue site. In some embodiments, the instillation regulator 155 may also be fluidly coupled to the negative-pressure source 105 through the dressing 110, as illustrated in the example of FIG. 1.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 105 may be combined with the controller 130 and other components into a therapy device 160.

In general, components of the therapy system 100 may be coupled directly or indirectly. For example, the negative-pressure source 105 may be directly coupled to the canister 115 and may be indirectly coupled to the dressing 110 through the canister 115. Coupling may include fluid, mechanical, thermal, electrical, or chemical coupling (such as a chemical bond), or some combination of coupling in some contexts. For example, the negative-pressure source 105 may be electrically coupled to the controller 130 and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 105, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 105 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The canister 115 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 130, may be a microprocessor or computer programmed to operate one or more components of the therapy system 100, such as the negative-pressure source 105. In some embodiments, for example, the controller 130 may be a microcontroller, which generally comprises an integrated circuit containing a processor core and a memory programmed to directly or indirectly control one or more operating parameters of the therapy system 100. Operating parameters may include the power applied to the negative-pressure source 105, the pressure generated by the negative-pressure source 105, or the pressure distributed to the tissue interface 120, for example. The controller 130 is also preferably configured to receive one or more input signals, such as a feedback signal, and programmed to modify one or more operating parameters based on the input signals.

Sensors, such as the first sensor 135 and the second sensor 140, may be any apparatus operable to detect or measure a physical phenomenon or property, and generally provide a signal indicative of the phenomenon or property that is detected or measured. For example, the first sensor 135 and the second sensor 140 may be configured to measure one or more operating parameters of the therapy system 100. In some embodiments, the first sensor 135 may be a transducer configured to measure pressure in a pneumatic pathway and convert the measurement to a signal indicative of the pressure measured. In some embodiments, for example, the first sensor 135 may be a piezo-resistive strain gauge. The second sensor 140 may optionally measure operating parameters of the negative-pressure source 105, such as a voltage or current, in some embodiments. Preferably, the signals from the first sensor 135 and the second sensor 140 are suitable as an input signal to the controller 130, but some signal conditioning may be appropriate in some embodiments. For example, the signal may need to be filtered or amplified before it can be processed by the controller 130. Typically, the signal is an electrical signal, but may be represented in other forms, such as an optical signal.

The tissue interface 120 can be generally adapted to partially or fully contact a tissue site. The tissue interface 120 may take many forms, and may have many sizes, shapes, or thicknesses, depending on a variety of factors, such as the type of treatment being implemented or the nature and size of a tissue site. For example, the size and shape of the tissue interface 120 may be adapted to the contours of deep and irregular shaped tissue sites. Any or all of the surfaces of the tissue interface 120 may have an uneven, coarse, or jagged profile.

In some embodiments, the tissue interface 120 may comprise or consist essentially of a manifold. A manifold in this context may comprise or consist essentially of a means for collecting or distributing fluid across or through the tissue interface 120 under pressure. For example, a manifold may be adapted to receive negative pressure from a source and distribute negative pressure through multiple apertures across or through the tissue interface 120, which may have the effect of collecting fluid from a tissue site and drawing the fluid toward the source. In some embodiments, the fluid path may be reversed or a secondary fluid path may be provided to facilitate delivering fluid, such as fluid from a source of instillation solution, to a tissue site.

In some illustrative embodiments, a manifold may comprise a plurality of pathways, which can be interconnected to improve distribution or collection of fluids. In some illustrative embodiments, a manifold may comprise or consist essentially of a porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

In some embodiments, the tissue interface 120 may comprise or consist essentially of reticulated foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The tensile strength of the tissue interface 120 may also vary according to needs of a prescribed therapy. For example, the tensile strength of foam may be increased for instillation of topical treatment solutions. The 25% compression load deflection of the tissue interface 120 may be at least 0.35 pounds per square inch, and the 65% compression load deflection may be at least 0.43 pounds per square inch. In some embodiments, the tensile strength of the tissue interface 120 may be at least 10 pounds per square inch. The tissue interface 120 may have a tear strength of at least 2.5 pounds per inch. In some embodiments, the tissue interface may be foam comprised of polyols such as polyester or polyether, isocyanate such as toluene diisocyanate, and polymerization modifiers such as amines and tin compounds. In some examples, the tissue interface 120 may be reticulated polyurethane foam such as found in GRANUFOAM™ dressing or V.A.C. VERAFLO™ dressing, both available from Kinetic Concepts, Inc. of San Antonio, Texas.

The thickness of the tissue interface 120 may also vary according to needs of a prescribed therapy. For example, the thickness of the tissue interface may be decreased to reduce tension on peripheral tissue. The thickness of the tissue interface 120 can also affect the conformability of the tissue interface 120. In some embodiments, a thickness in a range of about 5 millimeters to 10 millimeters may be suitable.

The tissue interface 120 may be either hydrophobic or hydrophilic. In an example in which the tissue interface 120 may be hydrophilic, the tissue interface 120 may also wick fluid away from a tissue site, while continuing to distribute negative pressure to the tissue site. The wicking properties of the tissue interface 120 may draw fluid away from a tissue site by capillary flow or other wicking mechanisms. An example of a hydrophilic material that may be suitable is a polyvinyl alcohol, open-cell foam such as V.A.C. WHITEFOAM™ dressing available from Kinetic Concepts, Inc. of San Antonio, Texas. Other hydrophilic foams may include those made from polyether. Other foams that may exhibit hydrophilic characteristics include hydrophobic foams that have been treated or coated to provide hydrophilicity.

In some embodiments, the tissue interface 120 may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and capralactones. The tissue interface 120 may further serve as a scaffold for new cell-growth, or a scaffold material may be used in conjunction with the tissue interface 120 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 125 may provide a bacterial barrier and protection from physical trauma. The cover 125 may also be constructed from a material that can reduce evaporative losses and provide a fluid seal between two components or two environments, such as between a therapeutic environment and a local external environment. The cover 125 may comprise or consist of, for example, an elastomeric film or membrane that can provide a seal adequate to maintain a negative pressure at a tissue site for a given negative-pressure source. The cover 125 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the cover 125 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 125 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 3M Company, Minneapolis Minnesota; polyurethane (PU) drape, commercially available from Avery Dennison Corporation, Pasadena, California; polyether block polyamide copolymer (PEBAX), for example, from Arkema S. A., Colombes, France; and Inspire 2301 and Inpsire 2327 polyurethane films, commercially available from Expopack Advanced Coatings, Wrexham, United Kingdom. In some embodiments, the cover 125 may comprise INSPIRE 2301 having an MVTR (upright cup technique) of 2600 g/m²/24 hours and a thickness of about 30 microns.

An attachment device may be used to attach the cover 125 to an attachment surface, such as undamaged epidermis, a gasket, or another cover. The attachment device may take many forms. For example, an attachment device may be a medically-acceptable, pressure-sensitive adhesive configured to bond the cover 125 to epidermis around a tissue site. In some embodiments, for example, some or all of the cover 125 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

The solution source 145 may also be representative of a container, canister, pouch, bag, or other storage component, which can provide a solution for instillation therapy. Compositions of solutions may vary according to a prescribed therapy, but examples of solutions that may be suitable for some prescriptions include hypochlorite-based solutions, silver nitrate (0.5%), sulfur-based solutions, biguanides, cationic solutions, and isotonic solutions.

In operation, the tissue interface 120 may be placed within, over, on, or otherwise proximate to a tissue site. If the tissue site is a wound, for example, the tissue interface 120 may partially or completely fill the wound, or it may be placed over the wound. The cover 125 may be placed over the tissue interface 120 and sealed to an attachment surface near a tissue site. For example, the cover 125 may be sealed to undamaged epidermis peripheral to a tissue site. Thus, the dressing 110 can provide a sealed therapeutic environment proximate to a tissue site, substantially isolated from the external environment, and the negative-pressure source 105 can reduce pressure in the sealed therapeutic environment.

The process of reducing pressure may be described illustratively herein as “delivering,” “distributing,” or “generating” negative pressure, for example. In general, exudate and other fluid flow toward lower pressure along a fluid path. Thus, the term “downstream” typically implies a location in a fluid path relatively closer to a source of negative pressure or further away from a source of positive pressure. Conversely, the term “upstream” implies a location relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications, such as by substituting a positive-pressure source for a negative-pressure source, and this descriptive convention should not be construed as limiting.

Negative pressure applied to the tissue site through the tissue interface 120 in the sealed therapeutic environment can induce macro-strain and micro-strain in the tissue site. Negative pressure can also remove exudate and other fluid from a tissue site, which can be collected in canister 115.

In some embodiments, the controller 130 may receive and process data from one or more sensors, such as the first sensor 135. The controller 130 may also control the operation of one or more components of the therapy system 100 to manage the pressure delivered to the tissue interface 120. In some embodiments, controller 130 may include an input for receiving a desired target pressure and may be programmed for processing data relating to the setting and inputting of the target pressure to be applied to the tissue interface 120. In some example embodiments, the target pressure may be a fixed pressure value set by an operator as the target negative pressure desired for therapy at a tissue site and then provided as input to the controller 130. The target pressure may vary from tissue site to tissue site based on the type of tissue forming a tissue site, the type of injury or wound (if any), the medical condition of the patient, and the preference of the attending physician. After selecting a desired target pressure, the controller 130 can operate the negative-pressure source 105 in one or more control modes based on the target pressure and may receive feedback from one or more sensors to maintain the target pressure at the tissue interface 120.

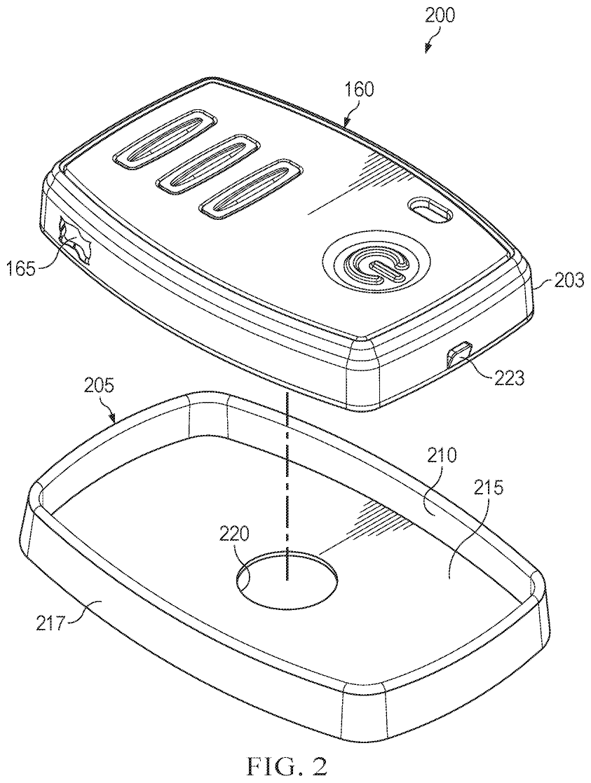

FIG. 2 is an exploded view of an example embodiment of a negative pressure therapy assembly 200 that may be associated with some embodiments of the therapy system 100 of FIG. 1. In some embodiments, the therapy assembly 200 includes the therapy device 160 and a mount 205. The therapy device 160 may include a housing 203 defining an enclosure 165 that is hermetically sealed. In some embodiments, the housing 203 may be rigid.

The mount 205 may be configured to receive the therapy device 160. For example, the mount 205 includes a receptacle 210 configured to receive at least a portion of the therapy device 160. The mount 205 also includes a mating surface 215 coupled to the receptacle 210. The mating surface 215 may be configured to create a hermetic seal with at least a portion of the therapy device 160. In some embodiments, the mating surface 215 and one or more walls 217 extending from an outer perimeter of the mating surface 215 define the receptacle 210. In some embodiments, an interior surface of one or more of the walls 217 may include an aperture or notch configured to receive a protrusion 223 on a side of the therapy device 160 and secure the therapy device 160 to the mount 205.

The mount 205 also includes at least one mount aperture 220 disposed in the mating surface 215. The at least one mount aperture 220 may be configured to be positioned in fluid communication with the therapy device 160.

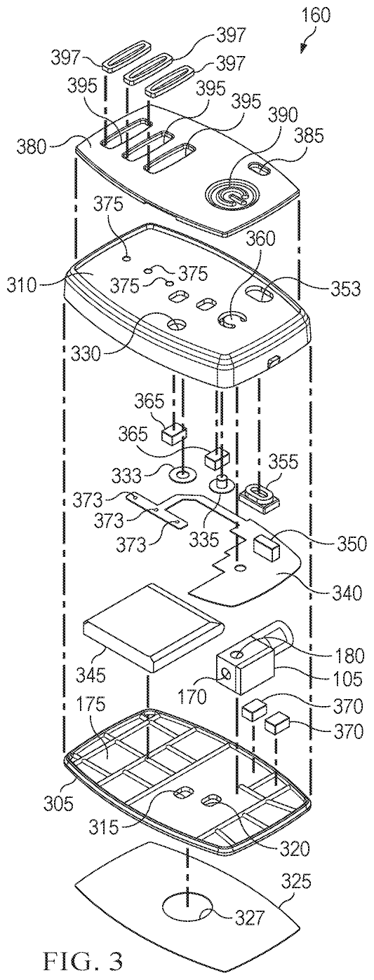

FIG. 3 is an exploded view of the therapy device 160 of FIG. 2, illustrating additional details that may be associated with some embodiments. In some embodiments, the housing 203 of the therapy device 160 includes a base portion 305 and a cover portion 310. The base portion 305 and the cover portion 310 are configured to be coupled and define the enclosure 165 shown in FIG. 2. The base portion 305 may include at least one housing aperture disposed in the base portion 305 and in fluid communication with the enclosure 165. For example, a first housing aperture 315 and a second housing aperture 320 may be disposed in the base portion 305 and configured to be in fluid communication with the enclosure. In some embodiments, the base portion 305 is substantially planar.

In some embodiments, a bottom seal 325 may be coupled to an exterior portion of the base portion 305. For example, the bottom seal 325 may be coupled to the base portion 305 opposite the cover portion 310. The bottom seal 325 may be vapor permeable and liquid impermeable. The bottom seal 325 may include a bottom aperture 327 disposed in the bottom seal 325 and configured to be fluidly coupled to the first housing aperture 315 and/or the second housing aperture 320. In some embodiments, a hydrophobic filter may be coupled to the bottom seal 325 adjacent the at least one housing aperture, such as the first housing aperture 315 and/or the second housing aperture 320, of the base portion 305. For example, the hydrophobic filter may be coupled to or associated with the bottom seal 325 adjacent the bottom aperture 327 in any suitable manner.

In some embodiments, the negative-pressure source 105, such as a negative pressure pump, is disposed in the enclosure 165 defined by the base portion 305 and the cover portion 310. The negative-pressure source 105 may be configured to expose the enclosure 165 to a negative pressure. In some embodiments, a reduced pressure inlet 170 of the negative-pressure source 105 may be in direct fluid communication with an internal wall 175 of the enclosure 165 and an exhaust outlet 180 of the negative-pressure source 105 may be in fluid communication with the ambient environment external to the enclosure 165. For example, the exhaust outlet 180 of the negative-pressure source 105 may be in fluid communication with the ambient environment through an exhaust aperture 330 disposed in the cover portion 310. In some embodiments, a seal, such an exhaust seal 333, may be coupled to the exhaust aperture 330.

In some embodiments, a valve 335 may be disposed in the enclosure 165 defined by the base portion 305 and the cover portion 310. The valve 335 may be configured to prevent overpressure in the enclosure 165. For example, the valve 335 may comprise a one-way valve configured to permit ingress of ambient air when the negative pressure in the enclosure 165 exceeds a threshold. In some embodiments, the valve 335 comprises an umbrella valve.

In some embodiments, one or more isolation mounts may be coupled to the negative-pressure source 105. For example, a first set of isolation mounts 365 may be configured to be coupled to a top portion of the negative-pressure source 105 and a second set of isolation mounts 370 may be configured to be coupled to a bottom portion of the negative-pressure source 105. Additionally, the first set of isolation mounts 365 may also be configured to be coupled to the cover portion 310 and the second set of isolation mounts 370 may be configured to be coupled to the base portion 305. The first set of isolation mounts 365 and the second set of isolation mounts 370 may be configured to reduce vibrations within the enclosure 165 while the negative-pressure source 105 is activated and operational.

Additionally, a printed circuit board (“PCB”) 340 may be disposed in the enclosure defined by the base portion 305 and the cover portion 310. In some embodiments, one or more of the negative-pressure source 105, a power source 345, and a communication port 350 may be coupled to the PCB 340. The power source 345 may be configured to supply power to the negative-pressure source 105. For example, the cover portion 310 may include a switch 360 configured to be electrically coupled to the PCB 340. The switch 360 may be configured to activate and deactivate the power source 345. In some embodiments, the power source 345 may be a battery, such as a rechargeable battery.

A communication aperture 353 may be disposed in the cover portion 310 and configured to expose the communication port 350 external to the enclosure. In some embodiments, a communication port seal 355 may be coupled to the communication aperture 353 and configured to seal the communication aperture 353 relative to the communication port 350. In some embodiments, the communication port 350 comprises a micro-USB port.

One or more indicator lights 373 may also be coupled to the PCB 340 and visible from the exterior of the enclosure. For example, a first set of indicator light apertures 375 may be disposed in the cover portion 310 and configured to expose the indicator lights 373. The power source 345 may be configured to supply power to the one or more indicator lights 373. In some embodiments, the one or more indicator lights 373 comprise light emitting diodes (“LEDs”).

In some embodiments, the therapy device 160 additionally includes a top seal 380 configured to be coupled to the cover portion 310. The top seal 380 may include a second communication aperture 385 configured to expose the communication aperture 353 and the communication port 350. The top seal 380 may also include a second switch 390 configured to be aligned with the switch 360 of the cover portion 310. Additionally, the top seal 380 may include a second set of indicator light apertures 395. The second set of indicator light apertures 395 may be configured to be aligned with the first set of indicator light apertures 375 in the cover portion 310 and to expose the one or more indicator lights 373. In some embodiments, one or more indicator covers 397 may be configured to be coupled to each of the second set of indicator light apertures 395 and cover the one or more indicator lights 373. The one or more indicator covers 397 may each comprise a different color to indicate a status of the therapy device 160. For example, the status may be one or more of an on condition, an alarm condition, an active charging condition, and a charging complete condition.

In operation, the therapy assembly 200 may be positioned adjacent a tissue site. For example, the mount 205 of the therapy assembly 200 may be configured to be coupled to the dressing 110. The mount 205 is configured to receive the therapy device 160 and fluidly couple the enclosure 165 of the therapy device 160 to the dressing 110 through one or more of the first housing aperture 315, the second housing aperture 320, the bottom aperture 327, and the mount aperture 220. Negative pressure generated by the negative-pressure source 105 within the enclosure 165 of the therapy device 160 may be delivered to the dressing 110.

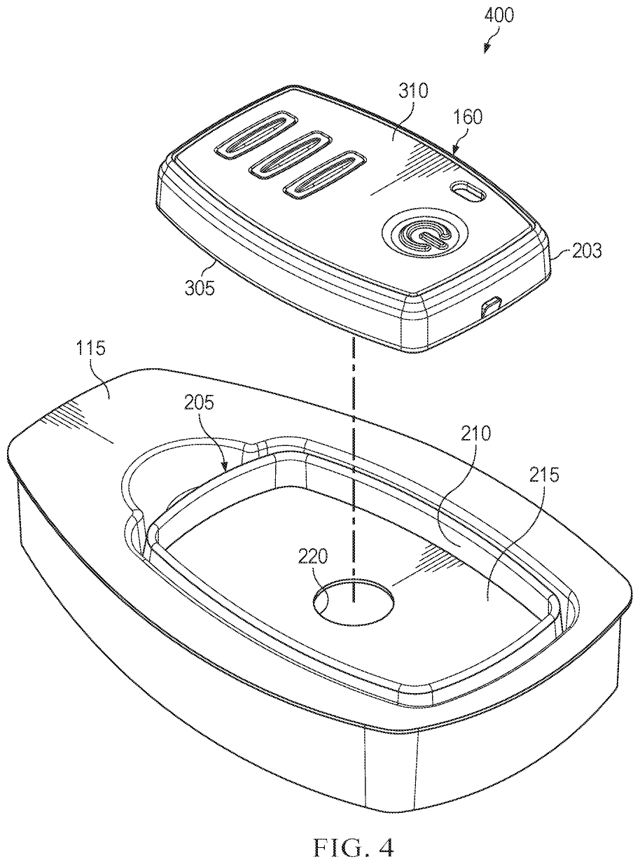

FIG. 4 is an exploded view of another example embodiment of a negative pressure therapy assembly 400 that may be associated with some embodiments of the therapy system 100 of FIG. 1. In some embodiments, the negative pressure therapy assembly 400 may include the therapy device 160 and the mount 205. The therapy device 160 and the mount 205 may be similar or analogous to the therapy device 160 and the mount 205 described with respect to FIGS. 2 and 3. For example, the mount 205 is configured to receive the therapy device 160 such that the mating surface 215 of the mount 205 creates a hermetic seal relative to at least a portion of the base portion 305 of the therapy device 160.

The negative pressure therapy assembly 400 may also include a canister, such as the canister 115. In some embodiments, the canister 115 is configured to receive the mount 205 and the therapy device 160. In other embodiments, the mount 205 may be coupled to the canister 115 and configured to receive the therapy device 160. For example, without limitation, the mount 205 may be coupled to the canister 115 by radio-frequency welding, ultrasonic welding, snap-fit, or a pressure-sensitive adhesive. The canister 115 may be configured to be fluidly coupled to a tissue site. For example, the canister 115 may be fluidly coupled to the dressing 110 positioned at the tissue site. In operation, reduced pressure from the therapy device 160 may be communicated to the tissue site though the canister 115 as shown in FIG. 1. As reduced pressure is supplied, fluids, such as wound exudate, may be removed from the tissue site and drawn into the canister 115

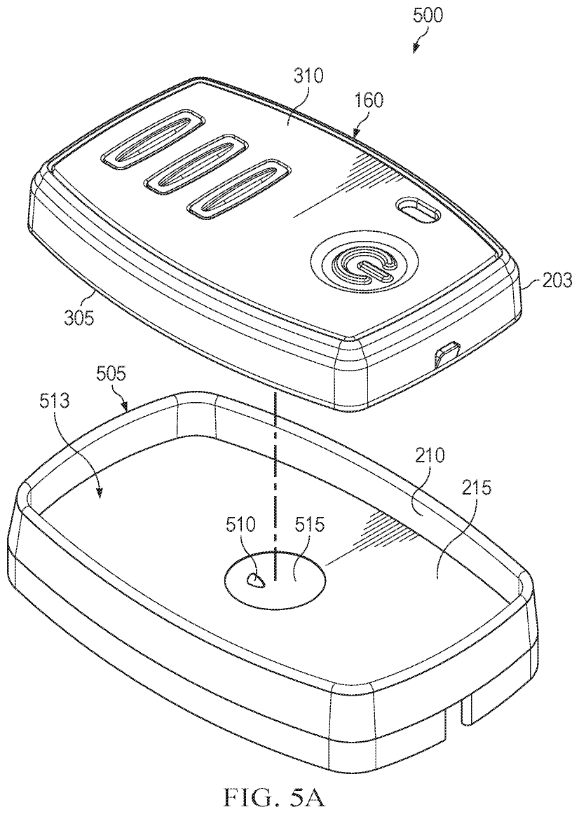

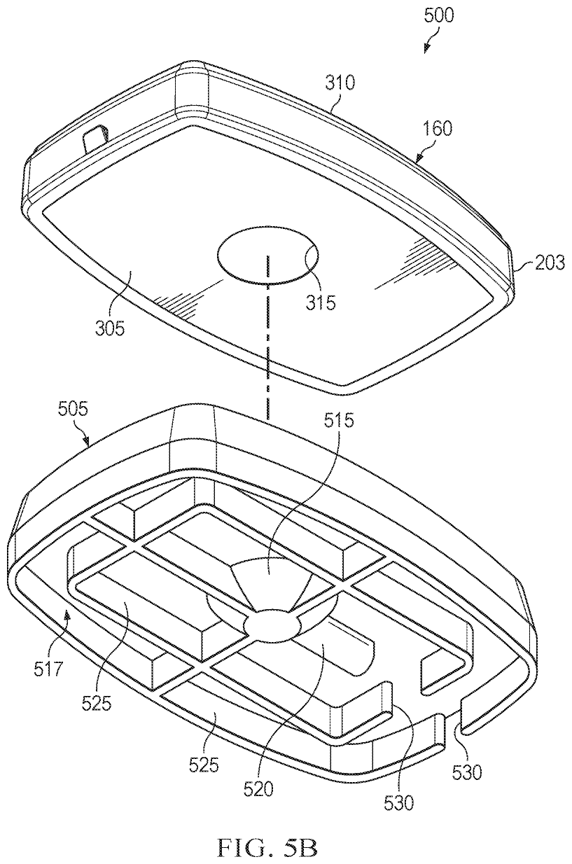

FIG. 5A is an exploded view of another example embodiment of a negative pressure therapy assembly 500 that may be associated with some embodiments of the therapy system 100 of FIG. 1. The negative pressure therapy assembly 500 may include the therapy device 160 and a mount 505. The therapy device 160 and the mount 505 may be similar or analogous to the therapy device 160 and the mount 205 described with respect to FIGS. 2 and 3.

The mount 505 may include the mating surface 215 and the receptacle 210 for receiving the therapy device 160. For example, the mating surface 215 of the mount 505 may be configured to create a hermetic seal relative to at least a portion of the base portion 305 of the therapy device 160. The mount 505 may also include an aperture 510 disposed in the mating surface 215. In some embodiments, the aperture 510 may be disposed in a recess 515 within the mating surface 215 of the mount 505.

With Reference to FIG. 5B, illustrating a bottom, exploded view of the negative pressure therapy assembly 500 of FIG. 5A, the aperture 510 may be configured to be fluidly coupled to an aperture in the base portion 305 of the therapy device 160. For example, the aperture 510 may be fluidly coupled to the enclosure 165 of the therapy device 160 through one or more of the first housing aperture 315, the second housing aperture 320, and the bottom aperture 327. The aperture 510 may extend from the mating surface 215 on a first side 513, shown in FIG. 5A, of the mount 505 to a second side 517 of the mount 505, shown in FIG. 5B. In some embodiments, the aperture 510 may be fluidly coupled to a port 520 on the second side 517 of the mount 505. The port 520 may be configured to receive a tube or conduit and fluidly couple the tissue site to the therapy device 160 though the mount 505.

In some embodiments, the second side 517 of the mount 505 may include one or more stand-offs 525. The stand-offs 525 may be configured to prevent the mount 505 from collapsing on the tube or conduit fluidly coupled to the port 520. In some embodiments, the stand-offs 525 may include one or more openings 530 configured to provide a pathway for the tube or conduit to the port 520.

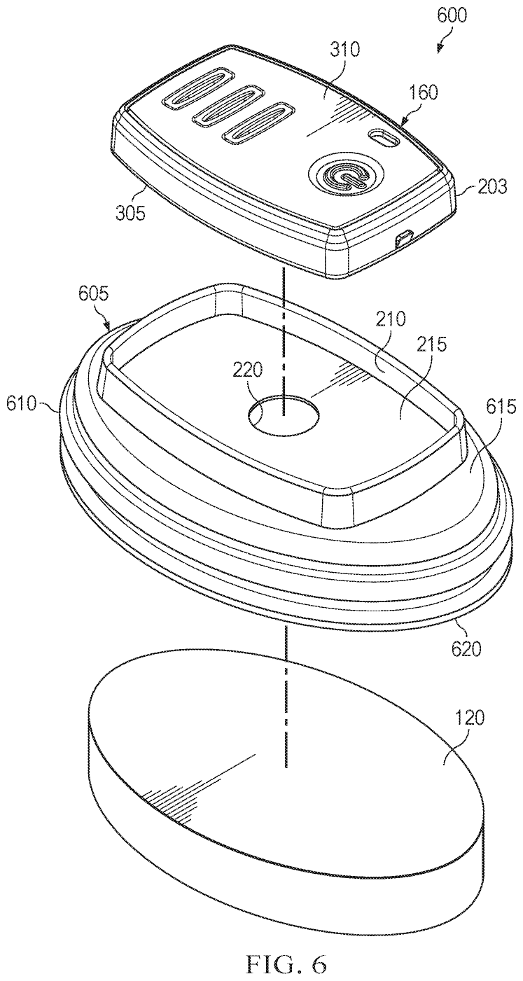

FIG. 6 is an exploded view of another example embodiment of a negative pressure therapy assembly 600 that may be associated with some embodiments of the therapy system 100 of FIG. 1. The negative pressure therapy assembly 600 may include the therapy device 160 and a mount 605. The therapy device 160 may be similar or analogous to the therapy device 160 described with respect to FIGS. 2 and 3.

The mount 605 may include the receptacle 210 configured to receive the therapy device 160, the mating surface 215 configured to create a hermetic seal relative to at least a portion of the base portion 305 of the therapy device 160, and a sealing member 610. In some embodiments, the sealing member 610 may comprise a first side 615 and a second side 620. The first side 615 of the sealing member 610 may form at least a portion of the mating surface 215 of the mount 605. The second side 620 of the sealing member 610 may form a second receptacle surrounded by walls of the sealing member 610 and configured to receive a manifold, such as the tissue interface 120. In operation, the tissue interface 120 may be placed within or over the tissue site and the sealing member 610 of the mount 605 may be placed over the tissue interface 120. The sealing member 610 is configured to seal the mount 605 over the tissue site. For example, the sealing member 610 may comprise a suction cup in some embodiments. The therapy device 160 is configured to supply reduced pressure to the tissue site through the mount aperture 220 and the tissue interface 120.

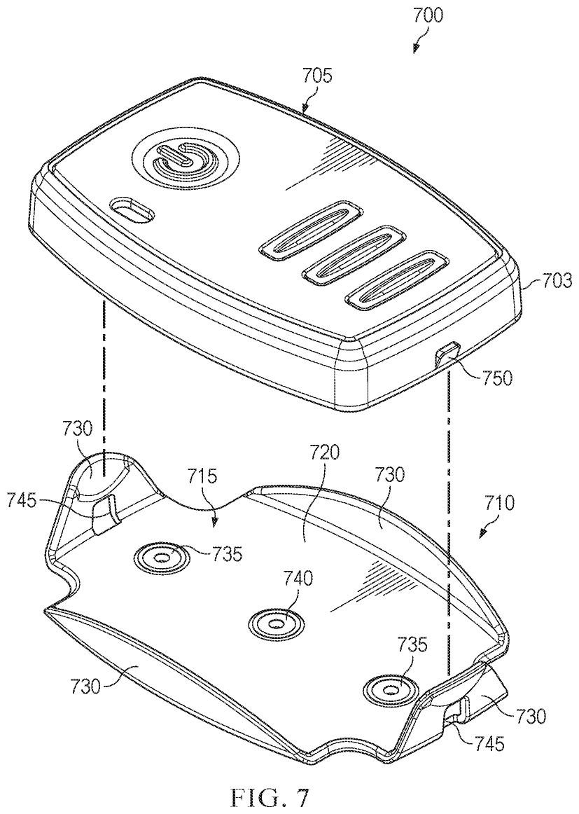

FIG. 7 is an exploded view of another example embodiment of a negative pressure therapy assembly 700 that may be associated with some embodiments of the therapy system 100 of FIG. 1. The therapy assembly 700 may include a therapy device 705 and a mount 710. In some embodiments, the therapy device 705 and the mount 710 may be similar or analogous to the therapy device 160 and the mount 205 described with respect to FIGS. 2 and 3.

In some embodiments, the mount 710 includes a receptacle 715 defined by a mating surface 720 and one or more walls 730 extending from the mating surface 720. The receptacle 715 of the mount 710 is configured to receive the therapy device 705. In some embodiments, one or more of the walls 730 of the mount 710 may include an aperture or notch, such as a notch 745. The notch 745 may be configured to receive a protrusion 750 on a side of the therapy device 705 and secure the therapy device 705 to the mount 710.

In some embodiments, the mount 710 also includes at least one vacuum port 735 and a sensing port 740 disposed in the mating surface 720. For example, the at least one vacuum port 735 may comprise two vacuum ports 735 and the sensing port 740 may be positioned equidistant from and between the two vacuum ports 735.

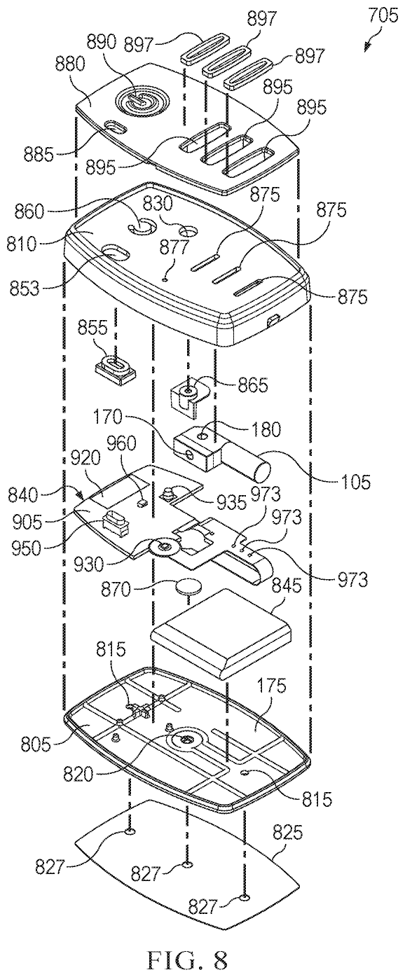

In some embodiments, the therapy device 705 may include a housing 703 that is hermetically sealed. With reference to FIG. 8, illustrating an exploded view of the therapy device 705 of FIG. 7, the housing 703 includes a base portion 805 and a cover portion 810. The base portion 805 and the cover portion 810 are configured to be coupled and define an enclosure analogous to the enclosure 165 shown in FIG. 2. The base portion 805 may be substantially planar in some embodiments. At least one vacuum port 815 and at least one sensing port 820 are also disposed in the base portion 805. For example, the at least one vacuum port 815 may comprise two vacuum ports 815. The at least one sensing port 820 may be positioned equidistant from and between the two vacuum ports 815.

In some embodiments, a bottom seal 825 is configured to be coupled to an exterior portion of the base portion 805. For example, the bottom seal 825 may be coupled to the base portion 805 opposite the cover portion 810. The bottom seal 825 may be vapor permeable and liquid impermeable. The bottom seal 825 may include a plurality of apertures 827 configured to be fluidly coupled to each of the vacuum ports 815 and the at least one sensing port 820. Additionally, at least one filter, such as a hydrophobic filter, may be coupled to each of the plurality of apertures 827.

In some embodiments, the negative-pressure source 105, such as a negative pressure pump, is disposed in the enclosure defined by the base portion 805 and the cover portion 810. The negative-pressure source 105 is configured to expose the enclosure to a negative pressure. In some embodiments, the reduced pressure inlet 170 of the negative-pressure source 105 may be in direct fluid communication with the internal wall 175 of the enclosure and the exhaust outlet 180 of the negative-pressure source 105 may be in fluid communication with the ambient environment external to the enclosure. For example, the exhaust outlet 180 of the negative-pressure source 105 may be in fluid communication with the ambient environment through an exhaust aperture 830 disposed in the cover portion 810. In some embodiments, a seal may be disposed within the enclosure and coupled to the exhaust aperture 330.

One or more isolation mounts may be coupled to the negative-pressure source 105. For example, a first isolation mount 865 may be configured to be coupled to a top portion of the negative-pressure source 105 and a second isolation mount 870 may be configured to be coupled to a bottom portion of the negative-pressure source 105. Additionally, the first isolation mount 865 may be configured to be coupled to the cover portion 310 and the second isolation mount 870 may be configured to be coupled to the base portion 305. The first isolation mount 865 and the second isolation mount 870 may be configured to reduce vibrations within the enclosure when the negative-pressure source 105 is activated and operational.

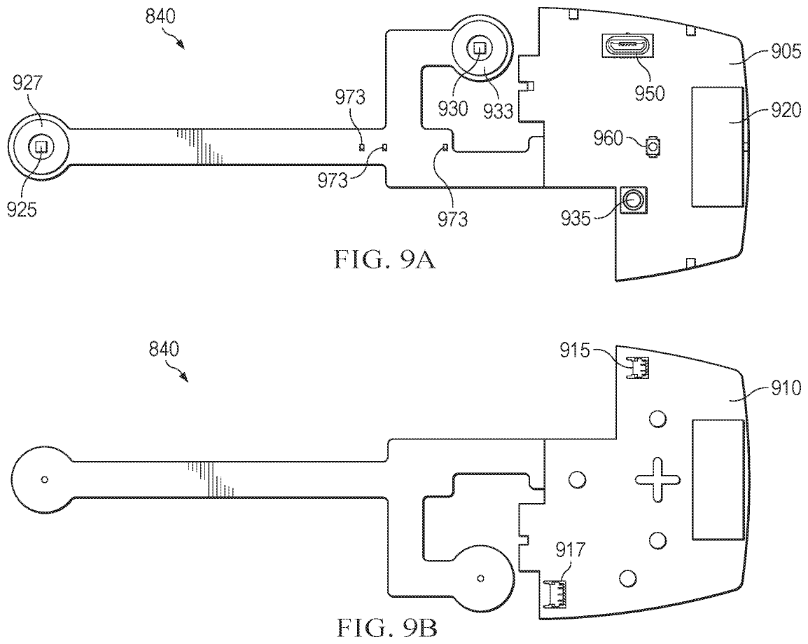

In some embodiments, a printed circuit board (“PCB”) 840 may be disposed in the enclosure defined by the base portion 805 and the cover portion 810. FIGS. 9A and 9B illustrate a top view and a bottom view, respectively, of an exemplary embodiment of the PCB 840. For example, the PCB 840 may include a first side 905 and a second side 910 opposite the first side 905. In some embodiments, one or more of the negative-pressure source 105, a power source 845, and a communication port 950 may be coupled to the PCB 340. The negative- pressure source 105 may be configured to be coupled to a first connector 915 on the second side 910 of the PCB 840. The power source 845 may be configured to be coupled to a second connector 917 on the second side 910 of the PCB 840 and to supply power to the negative-pressure source 105. For example, the PCB 840 may include a power switch 960 configured to be electrically coupled to the power source 845. The power switch 960 may be configured to activate and deactivate the power source 845. In some embodiments, the power source 845 may be a battery, such as a rechargeable battery.

With reference to FIGS. 8 and 9A, the cover portion 810 may include a switch 860 configured to be aligned with the power switch 960 of the PCB 840. In some embodiments, the switch 860 may be electrically coupled to the power switch 960 and configured to be depressed such that the switch 860 contacts the power switch 960 to activate and deactivate the power source 845.

In some embodiments, the communication port 950 may be coupled to the first side 905 of the PCB 840. With reference to FIG. 8, the cover portion 810 may include a communication aperture 853 configured to expose the communication port 950 external to the enclosure. A communication port seal 855 may also be coupled to the communication aperture 853 and configured to seal the communication aperture 853 relative to the communication port 850. In some embodiments, the communication port 850 comprises a micro-USB port.

With reference again to FIGS. 8 and 9A, one or more indicator lights 973 may be coupled to the first side 905 of the PCB 340 and visible from the exterior of the enclosure. For example, a first set of indicator light apertures 875 may be disposed in the cover portion 810 and configured to expose the indicator lights 973. The power source 845 may be configured to supply power to the one or more indicator lights 973. In some embodiments, the one or more indicator lights 973 comprise light emitting diodes.

In some embodiments, the PCB 840 also includes a transmitter and receiver 920 coupled to the first side 905. The transmitter and receiver 920 may be configured to communicate with an external device, such as a smartphone, a computer, and/or a smart dressing. For example, the transmitter and receiver 920 may be configured to communicate with the external device via Bluetooth and/or Wi-Fi. In some embodiments, the transmitter and receiver 920 comprises a Bluetooth antenna.

In some embodiments, one or more pressure transducers may be coupled to the PCB 840. For example, a first pressure transducer 925 may be coupled to at least a portion of the PCB 840. The first pressure transducer 925 may be configured to be fluidly coupled to the sensing port 820. A first seal 927 may be configured to couple the first pressure transducer 925 to the PCB 840 and to at least a portion of the base portion 805 adjacent the sensing port 820. For example, the first seal 927 may comprise a double-sided adhesive tape. In some embodiments, the therapy device 705 is configured to be positioned adjacent a tissue site and the first pressure transducer 925 is configured to read a pressure at the tissue site through the sensing port 820.

In some embodiments, a second pressure transducer 930 may be coupled to the PCB 840 and configured to read an atmospheric pressure external to the enclosure of the therapy device 705. For example, the second pressure transducer 930 may be fluidly coupled to an aperture 877 in the cover portion 810. A second seal 933 may be configured to couple the second pressure transducer 930 to the PCB 840 and to at least a portion of the cover portion 810 adjacent the aperture 877. The second seal 933 may also provide a fluid seal between the enclosure and the external environment. In some embodiments, the second seal 933 may comprise a double-sided adhesive tape.

Additionally, a third pressure transducer 935 may be configured to be coupled to the PCB 840 and configured to read the pressure within the enclosure. For example, the third pressure transducer 935 may be coupled to the first side 905 of the PCB 840.

Referring again to FIG. 8, the therapy device 705 may also include a top seal 880 configured to be coupled to the cover portion 810. The top seal 880 may include a second communication aperture 885 configured to expose the communication aperture 853 and the communication port 950. The top seal 880 may also include a second switch 890 configured to be aligned with the switch 860. Additionally, the top seal 880 may include a second set of indicator light apertures 895. The second set of indicator light apertures 895 may be configured to be aligned with the first set of indicator light apertures 875 and to expose the one or more indicator lights 873. In some embodiments, one or more indicator covers 897 may be configured to be coupled to each of the second set of indicator light apertures 895 and cover the one or more indicator lights 873. The one or more indicator covers 897 may each comprise a different color to indicate a status of the therapy device 160.

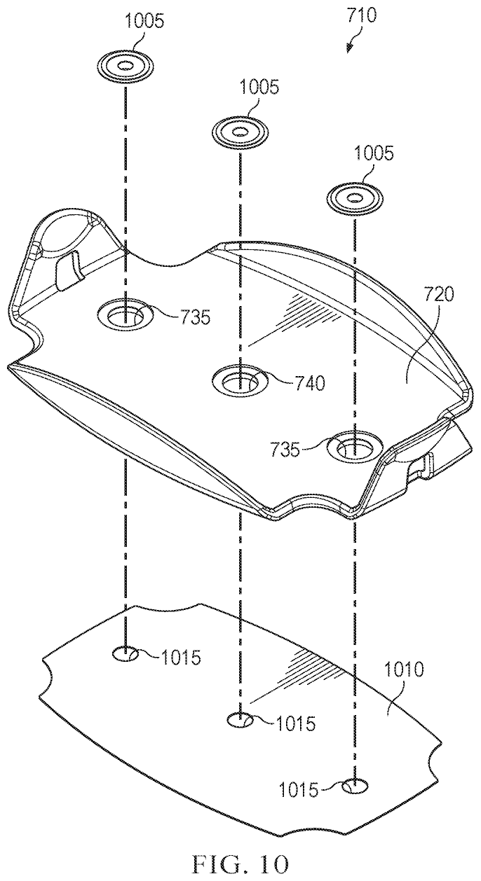

FIG. 10 is an exploded view of the mount 710 of the negative pressure therapy assembly 700 of FIG. 7, illustrating additional details that may be associated with some embodiments. The mount 710 may comprises a rigid thermoplastic polymer in some embodiments. The mount 710 may also include a plurality of sealing members 1005 configured to be coupled to each of the at least one vacuum port 735 and the sensing port 740. In some embodiments, the plurality of sealing members 1005 comprise a thermoplastic elastomer. In some additional embodiments, the mount 710 and the sealing members 1005 may be molded independently and the sealing members 1005 coupled to the at least one vacuum port 735 and the sensing port 740 of the mount 710. In other embodiments, the mount 710 and the scaling members 1005 may be molded together in a two-shot molding process.

In some embodiments, the mount 710 also includes a scaling layer 1010 configured to be coupled to the mount 710 opposite the mating surface 720. One or more apertures 1015 may be disposed in the sealing layer 1010. For example, each of the apertures 1015 may be configured to correspond with each of the at least one vacuum port 735 and the sensing port 740 disposed in the mount 710. In some embodiments, a filter, such as a hydrophobic filter, may be coupled to each of the apertures 1015. The filter may be configured to prevent fluid ingress into the therapy device 705 through the mount 710.

In operation, the therapy assembly 700 may be positioned adjacent a tissue site. For example, the mount 710 of the therapy assembly 700 may be configured to be coupled to the dressing 110. The mount 710 is configured to receive the therapy device 705 and fluidly couple the enclosure of the therapy device 705 to the dressing 110 through one or more of the vacuum ports 815, the sensing port 820, the apertures 827, the vacuum ports 735, the sensing port 740, and the apertures 1015. Negative pressure generated by the negative-pressure source 105 within the enclosure of the therapy device 705 may be delivered to the dressing 110. For example, negative pressure may be delivered to the dressing 110 from the enclosure through the vacuum ports 815.

In some embodiments, the therapy device 705 is configured to monitor the negative pressure delivered to the tissue site. For example, the first pressure transducer 925 may be fluidly coupled to the dressing 110 at the tissue site via the sensing port 820 of the base portion 805 of the therapy device 705 and the sensing port 740 of the mount 710. The first pressure transducer 925 may be configured to monitor the pressure at the tissue site such that a desired pressure may be achieved.

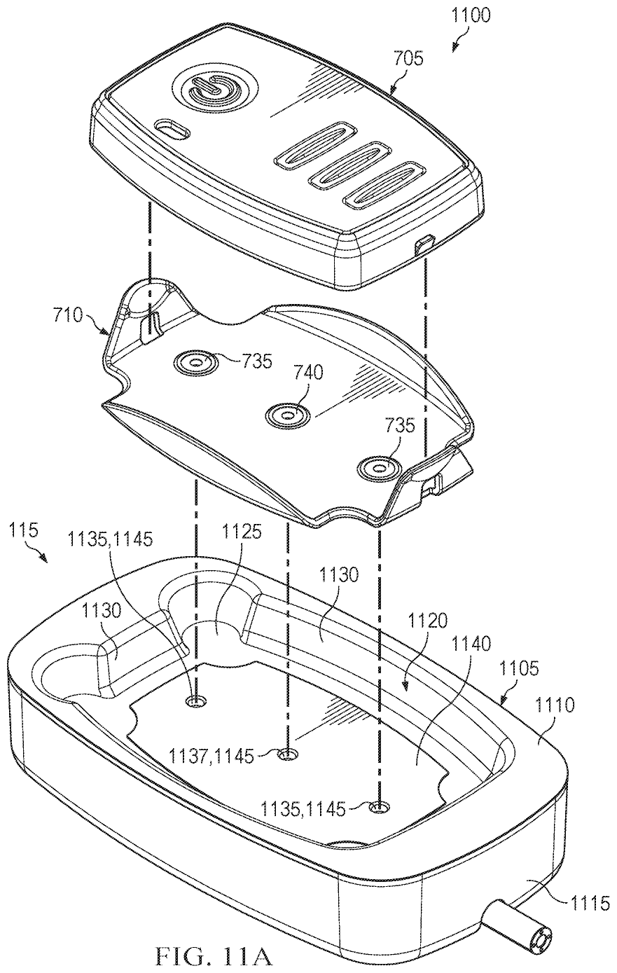

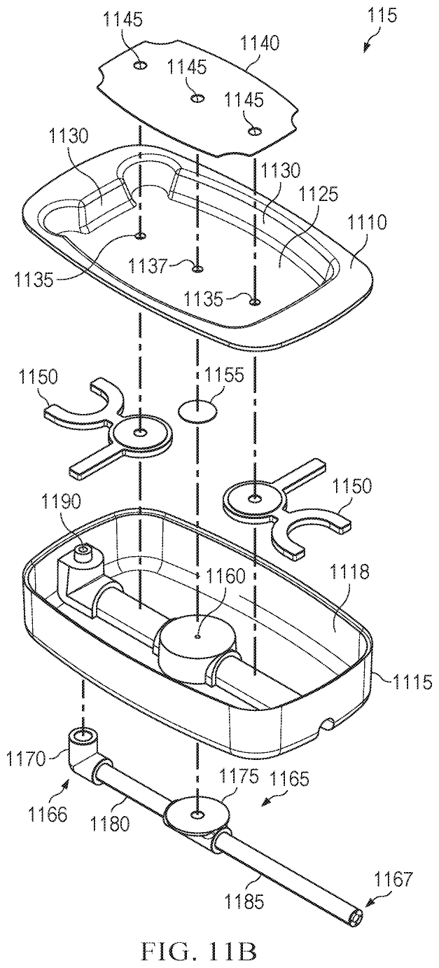

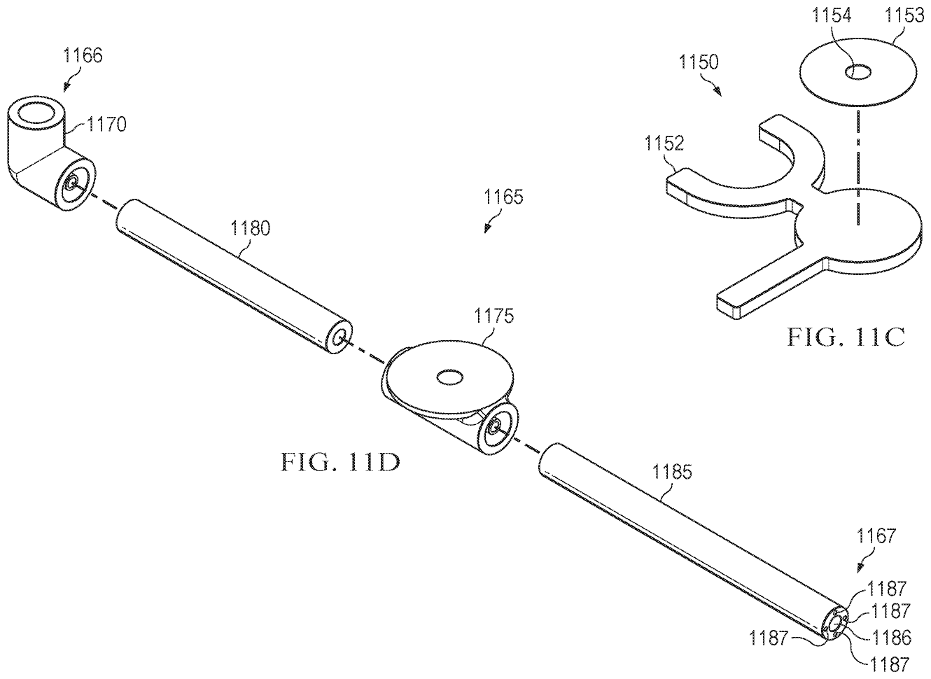

FIG. 11A is an exploded view of another example embodiment of a negative pressure therapy assembly 1100 that may be associated with some embodiments of the therapy system 100 of FIG. 1. In some embodiments, the therapy system 1100 includes the therapy device 705, the mount 710, and a canister, such as the canister 115. The therapy device 705 and the mount 710 may be similar or analogous to those discussed with reference to FIGS. 7-10.

With reference to FIGS. 11A and 11B, the canister 115 includes a canister housing 1105 comprising a top portion 1110 and a bottom portion 1115. The top portion 1110 and the bottom portion 1115 are configured to be coupled and form a canister enclosure 1118 shown in the exploded view of FIG. 11B.

In some embodiments, the top portion 1110 may include a canister receptable 1120 configured to receive the mount 710 and the therapy device 705. The canister receptacle 1120 may be formed by a mating surface 1125 and one or more walls 1130 extending from the mating surface 1125. In some embodiments, a plurality of apertures is disposed in the mating surface 1125 of the top portion 1110. For example, at least two vacuum ports 1135 and a sensing port 1137 may be disposed in the mating surface 1125. The vacuum ports 1135 may be configured to be fluidly coupled to the vacuum ports 735 of the mount 710 and the vacuum ports 815 of the therapy device 705 when the mount 710 and the therapy device 705 are positioned in the canister receptacle 1120 of the canister 115. In some embodiments, the mount 710 may be configured to be coupled to the mating surface 1125 with an adhesive 1140. The adhesive 1140 may comprise a double-sided adhesive in some embodiments. The adhesive 1140 may also include a plurality of apertures 1145. Each of the plurality of apertures 1145 may be configured to be fluidly coupled with the vacuum ports 735 and the sensing port 740 of the mount 710.

In some embodiments, the canister enclosure 1118 is configured to be fluidly coupled to the therapy device 705. For example, the negative-pressure source 105 of the therapy device 705 may be fluidly coupled to the canister enclosure through the vacuum ports 1135. The positioning of the vacuum ports 1135 on opposite sides of the sensing port 1137 allows negative pressure to be delivered to the tissue site through the canister 115 in multiple orientations.

Additionally, at least two vacuum filters 1150 may disposed in the canister enclosure 1118. The vacuum filters 1150 are configured to be coupled to each of the vacuum ports 1135 and to prevent fluid from exiting the canister enclosure 1118 through the vacuum ports 1135. With reference to FIG. 11C, each of the vacuum filters 1150 may comprise a filter portion 1152 and an adhesive 1153. In some embodiments, the filter portion 1152 comprises a foam. The adhesive 1153 is configured to couple the vacuum filter 1150 to the top portion 1110 adjacent the vacuum ports 1135. The adhesive 1153 includes at least one aperture 1154 to allow fluid communication between the vacuum ports 1135 and the filter portion 1152 through the aperture 1154. In some embodiments, the adhesive 1153 comprises a double-sided, waterproof adhesive tape.

Referring again to FIG. 11B, a sensing port 1160 may be disposed in the bottom portion 1115 of the canister 115. The sensing port 1160 is configured to be fluidly coupled to the therapy device 705, such as to the first pressure transducer 925 of the therapy device 705, through the sensing port 1137. In some embodiments, a sensing port filter 1155 is disposed in the canister receptacle and configured to be coupled between the sensing port 1160 and the sensing port 1137. The sensing port filter 1155 is configured to be coupled to the sensing port 1137 and to prevent fluid from exiting the canister enclosure through the sensing port 1137 and/or the sensing port 1160.

With reference to FIGS. 11B and 11D, the canister 115 may also include a tubing assembly 1165 configured to fluidly coupled the canister 115 to the tissue site. In some embodiments, the tubing assembly 1165 is configured to be recessed into the bottom portion 1115, as shown in FIG. 11B, such that the tubing assembly 1165 is flush with an exterior portion of the bottom portion 1115. The tubing assembly 1165 includes a first end 1166 and a second end 1167. The first end 1166 of the tubing assembly 1165 may include a first connector 1170 configured to fluidly couple the tubing assembly 1165 to the canister 115. For example, the first connector 1170 may be fluidly coupled to a canister port 1190 disposed in the bottom portion 1115 of the canister 115. A first conduit 1180 may be configured to be fluidly coupled to the first connector 1170. In some embodiments, the first conduit 1180 is a single-lumen tube. In other embodiments, the first conduit 1180 is a multi-lumen tube.

In some embodiments, a second connector 1175 is configured to be fluidly coupled to the first conduit 1180 and the sensing port 1160 of the canister 115. The second connector 1175 is configured to separate a negative pressure pathway from a sensing pathway. For example, the second connector 1175 separates the negative pressure pathway through the first connector 1170 and the first conduit 1180 from the sensing pathway from the sensing port 1160. A second conduit 1185 is configured to be fluidly coupled to the second connector 1175 and the tissue site. In some embodiments, the second conduit 1185 comprises a multi-lumen tube. For example, as shown in FIG. 11D, the second connector 1175 may direct the negative pressure pathway through a central lumen 1186 of the second conduit 1185 and may direct the sensing pathway through one or more secondary lumens 1187 surrounding the central lumen 1186.

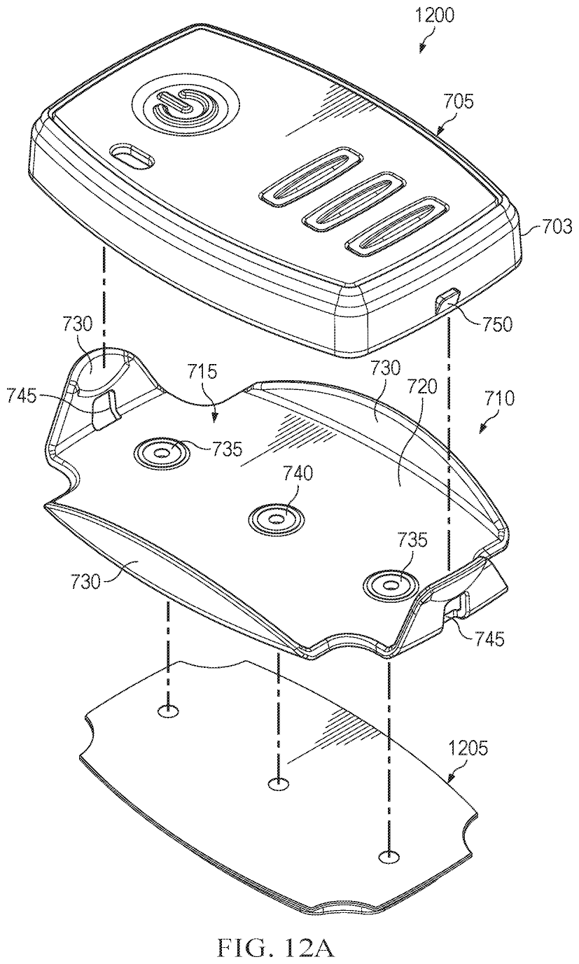

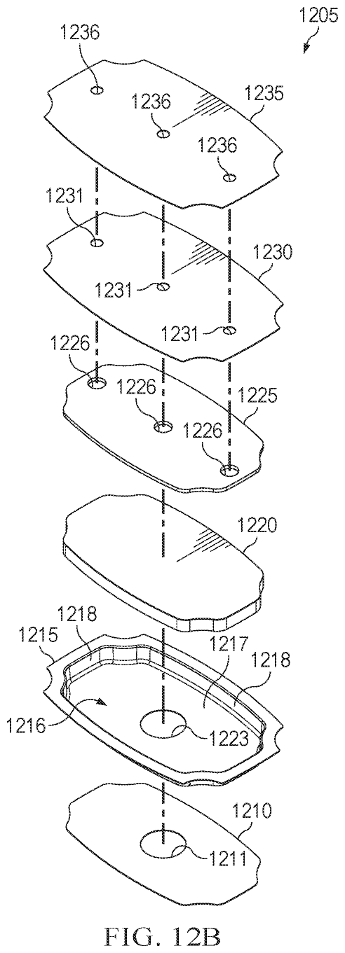

FIG. 12A is an exploded view of another example embodiment of a negative pressure therapy assembly 1200 that may be associated with some embodiments of the therapy system 100 of FIG. 1. In some embodiments, the therapy assembly 1200 includes the therapy device 705, the mount 710, and a dressing pad 1205. The therapy device 705 and the mount 710 may be similar or analogous to the therapy device 705 and the mount 710 discussed above with respect to FIGS. 7-10.

With Reference to FIG. 12B, illustrating an exploded view of the dressing pad 1205, the dressing pad 1205 may include a first adhesive layer 1210, a base layer 1215, a manifold 1220, a plate 1225, a film layer 1230, and a second adhesive layer 1235. In some embodiments, the base layer 1215 includes a receptacle 1216 formed by an interior surface and walls 1218 extending from the interior surface 1217. In some embodiments, the base layer 1215 comprises a polyurethane film. Additionally, at least one base layer aperture 1223 is disposed in the base layer 1215. The base layer aperture 1223 is configured to fluidly couple the receptacle 1216 of the base layer 1215 to the tissue site.

In some embodiments, the first adhesive layer 1210 is configured to be coupled to an exterior surface of the base layer 1215. A first adhesive aperture 1211 is disposed in the first adhesive layer 1210 and configured to be fluidly coupled to the base layer aperture 1223. The first adhesive layer 1210 is also configured to couple the dressing pad 1205 to a dressing, such as the dressing 110. For example, first adhesive layer 1210 may comprise a double-sided adhesive tape configured to be coupled to the base layer 1215 of the dressing pad 1205 and the dressing 110.

The manifold 1220 may be configured to be disposed in the receptacle 1216 of the base layer 1215. In some embodiments the manifold 1220 comprises an open cell foam. In some additional embodiments, the manifold 1220 comprises the same material discussed above with respect to the tissue interface 120. The plate 1225 is configured to be positioned over the manifold 1220. In some embodiments, the plate 1225 is configured to prevent the dressing pad 1205 and/or the mount 710 from flexing or bending under an application of negative pressure. The plate 1225 may also include a plurality of plate apertures 1226. The plurality of plate apertures 1226 are configured to be fluidly coupled to one or more of the vacuum ports 735 and the sensing port 740 in the mount 710.

The film layer 1230 is configured to be positioned over the plate 1225. In some embodiments, a perimeter of the film layer 1230 may be coupled to a perimeter of the base layer 1215. In such embodiments, the film layer 1230 and the base layer 1215 form an enclosure including the manifold 1220 and the plate 1225. A plurality of film apertures 1231 may be disposed in the film layer 1230. For example, each of the plurality of film apertures 1231 are configured to be fluidly coupled with the plurality of plate apertures 1226 and one or more of the vacuum ports 735 and the sensing port 740 in the mount 710.

In some embodiments, a second adhesive layer 1235 is configured to be coupled to the film layer 1230. The second adhesive layer 1235 may also be configured to be coupled to the mount 710. For example, the second adhesive layer 1235 may be a double-sided adhesive tape configured to couple the film layer 1230 of the dressing pad 1205 to the mount 710. A plurality of adhesive aperture 1236 may be disposed in the second adhesive layer 1235. Each of the plurality of adhesive apertures 1236 may be configured to fluidly coupled to the plurality of film apertures 1231, the plurality of plate apertures 1226, and one or more of the vacuum ports 735 and the sensing port 740 in the mount 710.

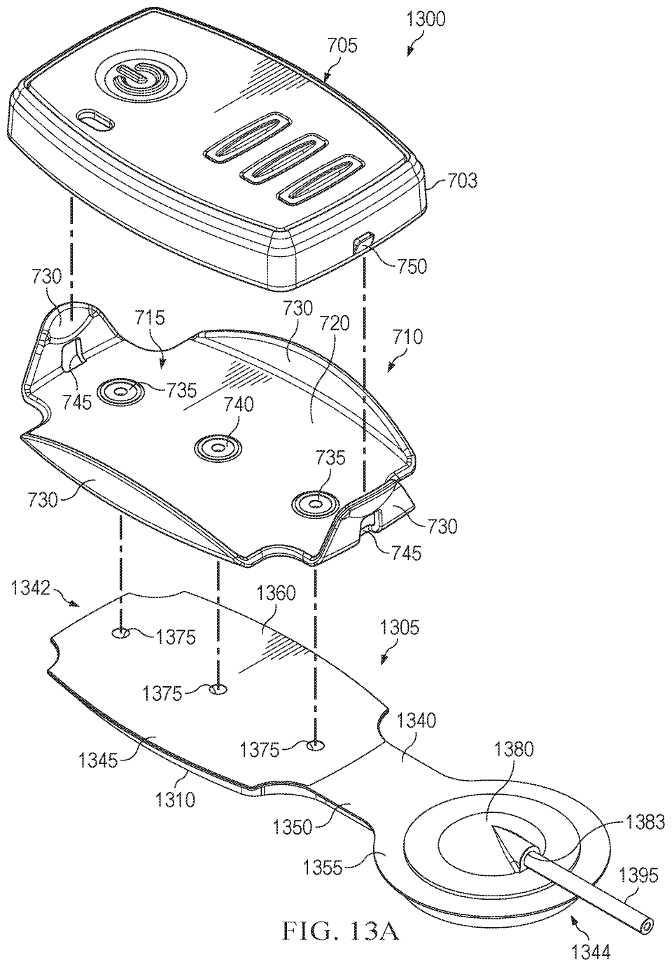

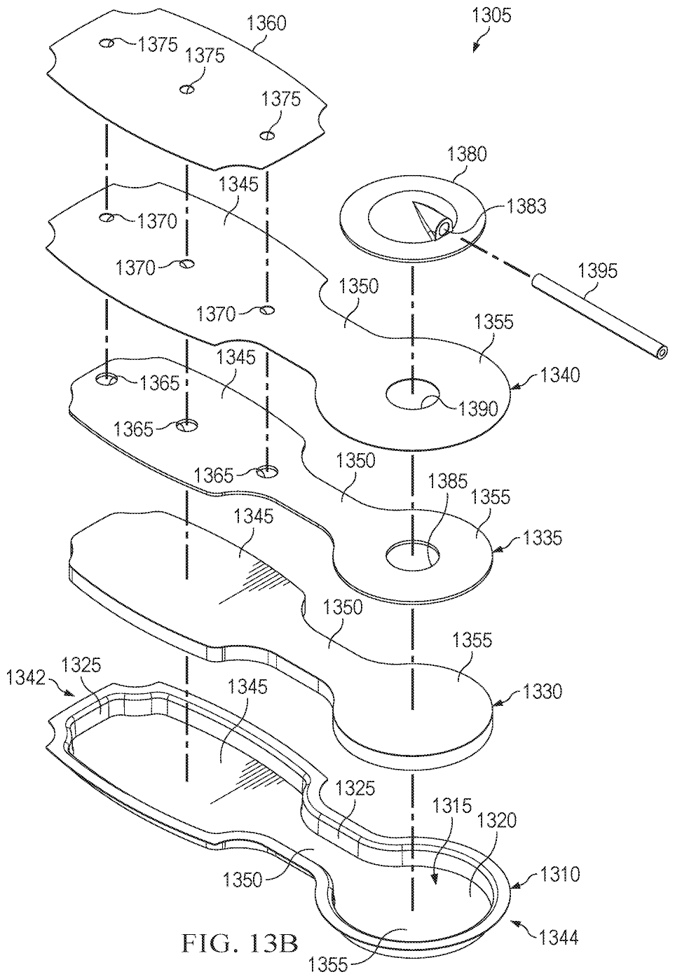

FIG. 13A is an exploded view of another example embodiment of a negative pressure therapy assembly 1300 that may be associated with some embodiments of the therapy system 100 of FIG. 1. In some embodiments, the therapy assembly 1300 includes the therapy device 705, the mount 710, and a dressing pad 1305. The therapy device 705 and the mount 710 may be similar or analogous to the therapy device 705 and the mount 710 discussed above with respect to FIGS. 7-10.

With reference to FIG. 13B, illustrating an exploded view of the dressing pad 1305, the dressing pad 1305 may include a base layer 1310. The base layer 1310 includes a receptacle 1315 defined by an interior surface 1320 and walls 1325 extending from the interior surface 1320. The walls 1325 may be formed around a perimeter of the interior surface 1320 of the base layer 1310. In some embodiments, the base layer 1310 comprises a polyurethane film.

In some embodiments, the dressing pad 1305 includes a manifold 1330. The manifold 1330 may be configured to be disposed in the receptacle 1315 of the base layer 1310. The dressing pad 1305 may also include a bolster plate 1335 adjacent the manifold 1330. For example, the bolster plate 1335 may be disposed in the receptacle 1315 of the base layer 1310 and positioned over the manifold 1330. In some embodiments, the bolster plate 1335 is configured to prevent the dressing pad 1305 and/or the mount 710 from flexing or bending under an application of negative pressure.

The dressing pad 1305 may also include a film layer 1340. The film layer 1340 may be configured to be positioned over the bolster plate 1335, the manifold 1330, and the base layer 1310. In some embodiments, at least a portion of the film layer 1340 is configured to be coupled to at least a portion of the base layer 1310 and form an enclosure. For example, a perimeter of the film layer 1340 may be configured to be coupled to the perimeter of the base layer 1310, such as the walls 1325, and form the enclosure. In such embodiments, one or both of the manifold 1330 and the bolster plate 1335 may be disposed in the enclosure.

In some embodiments, each of the base layer 1310, the manifold 1330, the bolster plate 1335, and the film layer 1340 may comprise the same shape. For example, each of the base layer 1310, the manifold 1330, the bolster plate 1335, and the film layer 1340 may comprise an elongate shape extending from a first side 1342 to a second side 1344 of the dressing pad 1305. Each of the base layer 1310, the manifold 1330, the bolster plate 1335, and the film layer 1340 may also comprise a first portion 1345, a second portion 1350, and third portion 1355. The first portion 1345 may be adjacent the first side 1342. In some embodiments, the first portion 1342 has a substantially rectangular shape. The third portion 1355 may be adjacent the second side 1344. In some embodiments, the third portion 1355 comprises a substantially circular shape. The second portion 1350 may be positioned between the first portion 1345 and the second portion 1350. In some embodiments, the second portion 1350 is narrower than the first portion 1345 and the third portion 1355. For example, the second portion 1350 may taper away from both the first portion 1345 and the second portion 1350.

In some embodiments, the dressing pad 1305 includes an adhesive layer 1360. The adhesive layer 1360 may be configured to be coupled to the first portion 1345 of the film layer 1340. The adhesive layer 1360 may also be configured to couple the dressing pad 1305 to the mount 710.

In some embodiments, each of the bolster plate 1335, the film layer 1340, and the adhesive layer 1360 may include a plurality of apertures. For example, the bolster plate 1335 may include a first plurality of apertures 1365, the film layer 1340 may include a second plurality of apertures 1370, and the adhesive layer 1360 may include a third plurality of apertures 1375. Each of the first, second, and third plurality of apertures 1365, 1370, 1375 are configured to be fluidly coupled to the vacuum ports 735 and the sensing port 740 of the mount 710. In such embodiments, the enclosure of the therapy device 705 may be fluidly coupled to the enclosure of the dressing pad 1305.

The dressing pad 1305 also includes a conduit interface 1380. The conduit interface 1380 may be configured to be coupled to at least a portion of the film layer 1340. For example, the conduit interface 1380 may be coupled to the third portion 1355 of the film layer 1340. The conduit interface 1380 is also configured to be fluidly coupled to the enclosure of the dressing pad 1305. For example, the conduit interface 1380 is configured to be fluidly coupled to the enclosure of the dressing pad 1305 through a first opening in the third portion 1355 of the bolster plate 1335 and a second opening 1390 in the third portion 1355 of the film layer 1340. The conduit interface 1380 may include a port 1383. The port 1383 is configured to receive a conduit 1395. The conduit 1395 may be configured to fluidly couple the dressing pad 1305 to a tissue site via the conduit interface 1380. Additionally, the therapy device 705 may be fluidly coupled to the tissue site through the dressing pad 1305 by the conduit interface 1380 and the conduit 1395. In some embodiments, the conduit 1395 comprises an absorbent material.

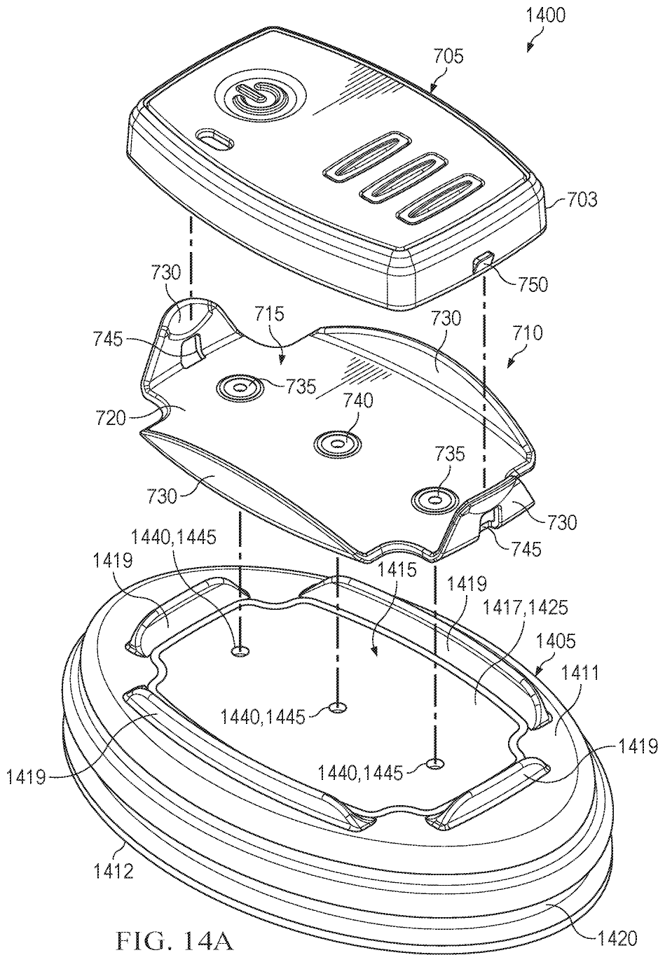

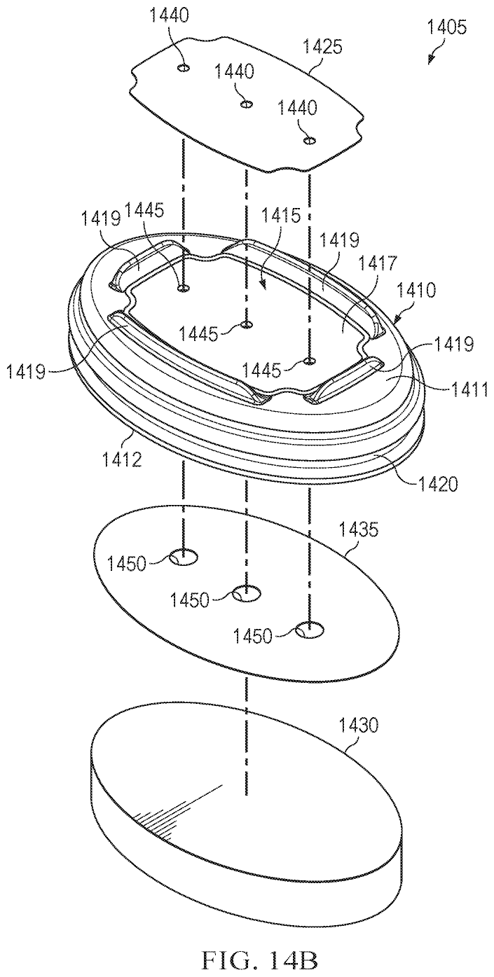

FIG. 14A is an exploded view of another example embodiment of a negative pressure therapy assembly 1400 that may be associated with some embodiments of the therapy system 100 of FIG. 1. In some embodiments, the therapy assembly 1400 includes the therapy device 705, the mount 710, and a trauma cup 1405. The therapy device 705 and the mount 710 may be similar or analogous to the therapy device 705 and the mount 710 discussed above with respect to FIGS. 7-10.

With reference to FIG. 14B, illustrating an exploded view of the trauma cup 1405, the trauma cup 1405 may include a cover portion 1410. The cover portion 1410 includes a first side 1411 and a second side 1412. The first side 1411 includes a first receptacle 1415 defined by a mating surface 1417 on the first side 1411 and one or more walls 1419. The walls 1419 extend from the first side 1411 of the cover portion and are configured to at least partially surround the mating surface 1417. In some embodiments, the walls 1419 comprise discrete members positioned adjacent each side of the mating surface 1417, as shown in FIGS. 14A-14B. In other embodiments, the walls 1419 may be a single, continuous member.

In some embodiments, a first adhesive layer 1425 may be coupled to the first side 1411 of the cover portion 1410. For example, the first adhesive layer 1425 may be coupled to the mating surface 1417. The first adhesive layer 1425 is configured to couple the trauma cup 1405 to the mount 710. In some embodiments, the first adhesive layer 1425 comprises a double-sided adhesive tape.

The second side 1412 of the cover portion 1410 includes a second receptacle defined by an extension 1420 extending from an outer perimeter of the second side 1412 of the cover portion 1410. The second receptacle may be configured to receive a manifold 1430. The manifold 1430 may be similar or analogous to the tissue interface 120 discussed above with respect to FIG. 1. A second adhesive layer 1435 is configured to couple the manifold 1430 to the second side 1412 of the cover portion 1410. In some embodiments, the second adhesive layer 1435 comprises a double-sided adhesive tape. In operation, the second receptacle of the cover portion 1410 is configured to create a seal over the manifold 1430 and a tissue site. For example, the second receptacle of the cover portion 1410 may comprise a suction cup in some embodiments.

In some additional embodiments, a first plurality of apertures 1440 is disposed in the first adhesive layer 1425, a second plurality of apertures are disposed in the cover portion 1410, and a third plurality of apertures 1450 are disposed in the second adhesive layer 1435. Each of the first plurality of apertures 1440, the second plurality of apertures 1445, and the third plurality of apertures 1450 may be configured to be fluidly coupled to the vacuum port 735 and the sensing port 740 of the mount 710. For example, the first plurality of apertures 1440, the second plurality of apertures 1445, and the third plurality of apertures 1450 may be configured to fluidly couple the therapy device 705 to the manifold 1430 positioned at the tissue site.

The systems, apparatuses, and methods described herein may provide significant advantages. For example, the therapy system 100 provides a therapy device with a reduced internal volume and size. The smaller size of the therapy device enables it to be mounted directly on a dressing or other device, such as a canister or blunt force trauma cup, which may also offer portability benefits.

The therapy system 100 also includes a universal mount for mounting the therapy device to the dressing or other device. For example, the therapy device may be positioned on or within the mount in multiple orientations. The mount includes at least one aperture for fluidly coupling the therapy device to the tissue site. For example, negative pressure may be delivered to the tissue site from the therapy device through the aperture in the mount. One or more pressure sensors within the therapy device may also be configured to measure the negative pressure at the tissue site through the aperture in the mount.

Moreover, the therapy device can be configured to communicate with other devices, such as smartphones. For example, the therapy device may be enabled with Bluetooth or Wi-Fi for such communication.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the dressing 110, the container 115, or both may be eliminated or separated from other components for manufacture or sale. In other example configurations, the controller 130 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. A negative pressure therapy assembly for treating a tissue site, comprising:
a negative pressure therapy device, comprising:
a housing defining an enclosure that is hermetically sealed, the housing including a base portion and a cover portion;
at least one housing aperture disposed through the base portion in fluid communication with the enclosure; and
a negative pressure pump disposed in the enclosure and configured to expose the enclosure to a negative pressure.

2. The negative pressure therapy assembly of claim 1, wherein the housing further comprises:
a bottom seal configured to be coupled to the base portion; and
a top seal configured to be coupled to the cover portion.

3. The negative pressure therapy assembly of claim 2, further comprising a hydrophobic filter coupled to the bottom seal adjacent the at least one housing aperture of the base portion.

4. (canceled)

5. The negative pressure therapy assembly of claim 1, wherein the housing is rigid and the base portion is substantially planar.

6. (canceled)

7. The negative pressure therapy assembly of claim 1, wherein a reduced pressure inlet of the negative pressure pump is in direct fluid communication with an internal wall of the enclosure, and wherein an exhaust outlet of the reduced pressure pump is in fluid communication with ambient environment external to the enclosure.

8. (canceled)

9. The negative pressure therapy assembly of claim 1, wherein the enclosure further comprises a valve configured to prevent overpressure in the enclosure.

10. (canceled)

11. The negative pressure therapy assembly of claim 1, wherein the enclosure further comprises:
a printed circuit board coupled to the pump; and
a power source coupled to the printed circuit board.

12. The negative pressure therapy assembly of claim 11, further comprising:
a communication port coupled to the printed circuit board;
a communication aperture disposed through the housing and positioned to expose the communication port external to the enclosure; and
a communication port seal configured to seal the communication aperture relative to the communication port.

13. (canceled)

14. The negative pressure therapy assembly of claim 1, wherein the enclosure further comprises:
a first set of isolation mounts configured to be coupled to a top portion of the pump; and
a second set of isolation mounts configured to be coupled to a bottom portion of the pump.

15. The negative pressure therapy assembly of claim 11, further comprising one or more indicator lights coupled to the printed circuit board and visible from exterior to the enclosure.

16-18. (canceled)

19. The negative pressure therapy assembly of claim 1, further comprising a mount configured to receive the negative pressure therapy device, wherein the mount comprises:
a receptacle configured to receive at least the base portion of the housing;
a mating surface coupled to the receptacle and configured create a hermetic seal relative to at least a portion of the base portion; and
at least one mount aperture disposed in the mating surface, each of the at least one mount apertures configured to be positioned in fluid communication with one of the at least one housing apertures.

20. A negative pressure therapy assembly for treating a tissue site, comprising:
a negative pressure therapy device, comprising:
a housing defining an enclosure that is hermetically sealed, the housing including a base portion and a cover portion;
at least one vacuum port disposed through the base portion in fluid communication with the enclosure;
at least one sensing port disposed in the base portion; and
a negative pressure pump disposed in the enclosure and configured expose the enclosure to a negative pressure.

21. The negative pressure therapy assembly of claim 20, further comprising:
a bottom seal configured to be coupled to the base portion; and
a top seal configured to be coupled to the cover portion.

22. The negative pressure therapy assembly of claim 21, further comprising at least one hydrophobic filter coupled to the bottom seal adjacent the at least one vacuum port and the at least one sensing port of the base.

23. (canceled)

24. The negative pressure therapy assembly of claim 20, wherein the enclosure comprises:
a printed circuit board coupled to the negative pressure pump and including a first pressure sensor in fluid communication with the sensing port, a second pressure sensor in fluid communication with the enclosure, and a third pressure sensor in fluid communication with ambient environment external to the enclosure; and
a power source coupled to the printed circuit board.

25. The negative pressure therapy assembly of claim 24, wherein the enclosure further comprises:
a first isolation mount coupled to a top portion of the negative pressure pump; and
a second isolation mount coupled to a bottom portion of the negative pressure pump.

26. The negative pressure therapy assembly of claim 24, further comprising:
a communication port coupled to the printed circuit board;
a communication aperture disposed through the housing and positioned to expose the communication port external to the enclosure; and
a communication port seal configured to seal the communication aperture relative to the communication port.

27. The negative pressure therapy assembly of claim 24, further comprising one or more indicator lights coupled to the printed circuit board.

28-30. (canceled)

31. The negative pressure therapy assembly of claim 20, further comprising a mount configured to receive the negative pressure therapy device, wherein the mount comprises:
a receptacle configured to receive at least the base portion of the housing;
a mating surface coupled to the receptacle and configured create a hermetic seal relative to at least a portion of the base portion; and
at least one mount aperture disposed in the mating surface, each of the at least one mount apertures configured to be positioned in fluid communication with one of the at least one vacuum port or one of the at least one sensing port.

32. The negative pressure therapy assembly of claim 31, wherein the at least one mount aperture comprises two vacuum apertures and one sensing aperture positioned equidistant from and between the two vacuum apertures, each of the two vacuum apertures configured to be fluidly coupled to one of the at least one vacuum ports and the sensing aperture configured to be fluidly coupled to the sensing port.

33. The negative pressure therapy assembly of claim 31, wherein the mount further comprises at least one aperture seal configured to be coupled to each of the at least one mounting apertures.

34. (canceled)

35. A system for treating a tissue site with negative pressure, the system comprising:
a negative pressure therapy device, comprising:
a housing defining an enclosure that is hermetically sealed, the housing including a base portion and a cover portion,
at least one vacuum port disposed through the base portion in fluid communication with the enclosure,
at least one sensing port disposed in the base portion, and
a negative pressure pump disposed in the enclosure and configured to expose the enclosure to the negative pressure;
a mount configured to receive the negative pressure therapy device, the mount comprising:
a receptacle configured to receive at least the base portion of the housing;
a mating surface coupled to the receptacle and configured create a hermetic seal relative to at least a portion of the base portion, and
at least one mount aperture disposed in the mating surface, each of the at least one mount apertures configured to be positioned in fluid communication with one of the at least one vacuum port or one of the at least one sensing port; and
a dressing configured to be positioned at the tissue and in fluid communication with the at least one mount aperture.

36. (canceled)
